(19) United States
(12) Patent Application Publication
(10) Pub. No.: US 2026/0355411 A1
(43) Pub. Date: Nov. 12, 2026

(54) ATTACHMENT SYSTEM FOR INJECTION MONITORING MODULE

(71) Applicant: BIOCORP PRODUCTION S.A.S., Issoire (FR)

(72) Inventor: Alain MARCOZ, Issoire (FR)

(21) Appl. No.: 19/486,963

(22) PCT Filed: Jul. 3, 2023
(86) PCT No.: PCT/IB2023/000416

§ 371 Date: Nov. 24, 2025

(51) Int. Cl. A61M 5/00; A61M 5/20

(52) U.S. Cl. CPC A61M 5/0091; A61M 5/20; A61M 2205/52; A61M 2209/082

(57) ABSTRACT

An injection monitoring module attachment system for mounting an injection monitoring module on an injection pen comprises a first annular body with an annular wall, and defining an inner central bore extending with a central axis. A second annular body is mounted coaxially on, and around, the first annular body and is configured to be movable relative to the first annular body, from a first position to a second position. In the first position, the second annular body exerts no inward radial force on the first annular wall. In the second position, the second annular body exerts an inward radial force on the first annular wall, thereby operating a reduction in the diameter of the first annular wall.

The present invention relates generally to monitoring systems for injectable drug delivery devices, and in particular to an injection monitoring module for for monitoring the end of an injection when using an injection pen system.

Injection monitoring is a well known field associated with injectable drug delivery devices, especially with regard to infusion systems, for example. Over time, such monitoring systems have been made available for injection pen systems for delivery of a drug, enabling users of such pen injection systems, and health care professionals involved in the treatment and follow-up of such patients, to monitor more closely the associated injection regimes, and in many cases, the doses actually administered, in an attempt to lead to better healthcare outcomes. These developments have been accompanied by the increased associated use of software and portable communications devices such as tablets or smartphones, which have been programmed to receive information from, and interact with, the monitoring systems in order to provide information to the user or healthcare professional on-the-fly, or at regular intervals via appropriate communications units included in the monitoring systems.

In regard to pen injection systems in particular, for example, one of the challenges has been to provide easy to use, reliable and fairly failsafe monitoring systems that can be adapted to the various different variants of such commercially available pen injection systems, of which there are many. Previous attempts at providing such monitoring systems have usually involved adapting the body of the pen injection system by including electronic components therein along with one or more sensors. Such adapted pen injection systems tend to be very specific to a given brand or a manufacturer, and thus of little or no use with pen injection devices of other manufacturers. There has furthermore been a tendency to attempt to reduce the overall volume of the injection pen bodies as much as possible through miniaturization of the complex electronic components, which in turn has brought about its own problems, in particular with regard to electromagnetic interference between the various components due to the close proximities of the circuits providing the required or desired integrated functionality. Moving the sensors in such monitoring systems further away from the source of electromagnetic interference only further complicates matters, potentially leading to erroneous readings, or requiring further systems to compensate for the physical separation of the sensors from the other electronic components, such as a microcontroller designed to control and command the various components and manage their interactions.

The injection pen systems in question are well known per se and are commonly equipped with a proximally located dose setting wheel and injection activator, the dose setting wheel being rotatable about a central longitudinal axis of the pen injection system. The wheel is rotated by the user to select the dose of drug to be administered. The pen is generally configured, either mechanically or electromechanically to effect an injection upon activation of an injection activator. Such injection activators are quite commonly a simple press or push-button, in mechanical or electrical contact with the dispensing mechanism located within the pen injection system, the pressing of which causes the injection mechanism to fire and inject the drug contained within the pen injection system. In some pen injector systems, the dose setting wheel is configured to rotate not only during dose setting, but also during injection. This is generally achieved through the inclusion of one or more metallic components, such as a helically wound drive spring located within a housing body of the injection pen system and physically coupled to the dose setting wheel. As such metallic elements are relatively large objects in comparison to the electronic component systems that are included in many pen injection systems today, these large metallic objects can further perturb signals that the sensors in such electronic component systems are designed to capture or pick up, rendering the systems potentially less accurate, and/or requiring that complex correction mechanisms be put in place to avoid calculation errors.

Commonly commercialized injection pen systems have a variety of pen body shapes and diameters, and as a general rule, each type of injection pen has a set of dimensions that are specifically tailored for that pen, or the medicament, drug or substance intended to be administered via such an injection pen system. Additionally, each pen manufacturer, for any given brand of pen, and/or associated injectable product, may have pen bodies which vary in dimensions, in particular, in outer diameter, i.e. the diameter of a virtual circle following the circumference of an outer peripheral surface of the pen body, due to manufacturing tolerances. This variation in injection pen body outer diameters can be problematic for injection monitoring modules with fixed diameter inner bores because, even with the provision of volume-adaptive internal coatings, such as push-fit elastomers, it can be difficult to obtain a securely snug fit of the injection monitoring module onto and around the outer surface of the injection pen body, or a dose setting wheel of such an injection pen system. Additionally, the frictional nature of the volume-adaptive elastomers provided on such injection monitoring modules and the thereby induced necessity for a certain degree of axial and rotational force to be applied by the user when mounting the injection monitoring module, can make it difficult for many users to either simply mount the injection monitoring module onto the injection pen, or to position such injection monitoring modules precisely on the injection pen. Such a situation can potentially lead to incorrect subsequent usage, and/or incorrect readings, such as, for example, an unwanted rotation of one or more parts of the injection monitoring module during mounting of the monitoring module onto the injection pen, leading to an erroneous detection of a measurable indicator or signal, such as a magnetic field.

As a result of the above, there remains the need to provide an injection monitoring module which is both easy to mount, and precisely position, onto a pen injection system, and similarly easy to dismount from the pen injection system, for example with users who might, in addition to the pathology requiring the administration of a medicament via an injection pen system, suffer from a range of physical or other disabilities. In such cases, having an injection monitoring module that is difficult to mount, and/or position correctly, for example requiring the application of a one-handed axial force to push the injection monitoring module into the correct position along the outer surface of the injection pen body, and/or dismount the injection monitoring module from the injection pen body by applying a one-handed pulling force in an opposite axial direction, may only serve to deter, or reduce observance of the treatment regime by the disabled user.

The applicant has previously described a number of injection monitoring modules for injection pen systems, for example, such as those published as WO2017013463A1, WO2019175615A1, WO2019175790A1, WO2020217076A1, WO2021140352A1, WO2020217094A1, WO2021260404A1, and WO2022079462A1.

As may be used in the present specification, the terms “pen injection system” and “injection pen system” are used interchangeably to designate a generally handheld pen-shaped injection system, such systems being readily well known per se and commercially available for use in the treatment of many various medical indications. These systems are also often generally designed for self-injection of a drug by the user in need of treatment for the given medical indication. This is for example the case with insulin, supplied in various forms for use in the treatment of diabetes, for example the pen injection systems commercialized under the brand names FlexPen®, as commercialized by Novo Nordisk, Kwikpen®, as commercialized Eli Lilly, or Lantus Solostar®, as commercialized by Sanofi, being but three of the most well known, but also with other hormones, such as growth hormone, or follicular stimulating hormones such as follitropin delta, commercialized in a pen injection system called Rekovelle®. Other drugs are also used with this category of medical devices, and may be required, for example, to address a number of potentially life-threatening situations, enabling immediate emergency injection of a required drug, such as anaphylactic shock treatments, anti-coagulants, opioid receptor agonists and antagonists, and the like, to the extent that it has become a common occurrence for patients suffering from, or susceptible to, such ailments to carry these devices around with them.

Additionally, the terms “proximal”, “proximally”, “distal” and “distally”, where such terms may be used in the present specification, refer to relative positions with regard to any of an injection monitoring system, injection monitoring module, and pen injection system in general, wherein “proximal” relates to a point or position or direction that is generally oriented in the direction towards the holder of the injection monitoring system, injection monitoring module, or pen injection system, and “distal” relates to a point or position or direction that is generally oriented in the direction away from the holder of the injection monitoring system, injection monitoring module, or pen injection system, for example towards a target site for injection, whether that be another part of the user's body, or a different person's, or animal's, body, or simply a target site for ejection of the substance contained within the pen injection system.

The injection pen system, to which the injection monitoring module according to the invention is adapted and configured for removable attachment, is generally equipped with a proximally located dose setting wheel and an injection actuator. The dose setting wheel rotates about a central longitudinal axis of the pen injection system to allow a user to set the dose of medicament for injection. During the dose setting, or dose “dialing” step, the dose setting wheel is generally rotatable in a clockwise, and generally also, a counter-clockwise direction, these directions corresponding respectively to either an increase in the selected dose, or a decrease in the selected dose to be administered, or vice-versa, depending on the manufacturer. The injection actuator is often represented by a push-button, usually located proximally of the dose setting wheel, and in the majority of injection pens at the proximal extremity of the injection pen system. After a dose has been set, or “dialed”, as the term is commonly known in the art, when a user of the injection system then presses the injection actuator in a distal direction, a piston is driven which is connected to a plunger in order to expel drug from a chamber within the injection pen body out through a needle that the user has inserted into an appropriate injection site, for example, the skin, fatty tissue, or muscle, depending on the type of drug to be administered. The dose setting wheel is sometimes, but not necessarily, also coupled to the injection drive mechanism so that it can, depending on the manufacturer and model of injection pen, also rotate as injection of the drug proceeds. The functioning of such injection systems is well known per se in the art. The monitoring module as envisaged according to the present invention is intended for mounting onto a pen injection system in which the dose setting wheel can be configured to either rotate during the ejection/injection phase of operation, or, on the contrary, not rotate during the ejection/injection phase of operation of the pen injection system. For example, the Kwikpen® injection pen mentioned above does not have a dose setting wheel that rotates during injection, whereas the dose setting wheel of the Lantus Solostar®, FlexPen® and Rekovelle® injection pens do rotate during injection.

Accordingly, one aspect of the present invention is to provide an attachment system configured and adapted for mounting an injection monitoring module on an injection pen comprising:
a first annular body having a length and a diameter, and comprising an annular wall extending along the length from a first end to a second end, the length being greater than the diameter, the annular wall defining an inner central bore extending along the length and having a central longitudinal axis;
a second annular body mounted coaxially on, and around, the second end of the first annular body;
the second annular body being configured to be movable relative to the first annular body, from a first position to a second position;
wherein, in the first position, the second annular body exerts no inward radial force on at least one portion of the annular wall of the first annular body, and
wherein, in the second position, the second annular body exerts an inward radial force on the at least one portion of the first annular wall,
wherein the inward radial force applied to the at least one portion of the first annular wall operates a reduction in the diameter of the at least one portion of the first annular wall.

It will be understood from the above that the first position and the second position relate to two different positions of the first annular body and the second annular body, one with respect to the other. In the first position, the second annular body is in direct or indirect physical, or surface-to-surface engaging, contact with the first annular body, but exerts no radially inward force on the first annular body. For example, in the first position, the second annular body may be attached, or mounted, to the first annular body in a manner that exerts no radially inwardly directed force, i.e. no force directed towards the central longitudinal axis. In the first position, for example, the second annular body can be configured to rotate freely about the central longitudinal axis, whilst nonetheless being mounted onto, or attached to, and engaging with, a portion of the first annular body.

It will also be understood from the above that second annular body exerts a radially inwardly directed force on the first annular body when the second annular body is moved from the first position into the second position. The radially inwardly exerted force causes a reduction in diameter of the at least one portion of the first annular wall, the reduction in diameter enabling a precise and secure positioning of the attachment system, along with any injection monitoring module mounted co-axially at least partially within the inner central bore of the attachment system, onto an injection pen, in particular, around a dose setting wheel portion of the injection pen.

According to another aspect, the annular wall of the first annular body comprises a plurality of elastically radially deformable fingers extending towards the second end. For example, the annular wall of the first annular body may comprise 2 or more elastically radially deformable fingers, such as 3, 4, 5, 6, 7, 8 or more, elastically radially deformable fingers, extending towards the second end of the annular wall of the first annular body. The fingers may extend from any suitable point along a length of the first annular wall towards the second end of the first annular wall body.

According to another aspect, the plurality of elastically radially deformable fingers are distributed radially about the central longitudinal axis, and preferably are distributed radially equally about the central longitudinal axis.

According to another aspect, the elastically radially deformable fingers are deformable radially inwardly toward the central longitudinal axis, when the second annular body exerts an inward radial force on the at least one portion of the first annular wall.

According to another aspect, each finger of the plurality of elastically radially deformable fingers has a portion of inward-facing surface configured to provide surface-engaging contact with an outer peripheral surface of an injection pen body, and preferably a dose-setting wheel portion of the injection pen body, when the attachment system is mounted onto an injection pen, and the second annular body is in the second position. It will be understood here that the surface-engaging contact portion of the inward-facing surface of each elastically deformable finger can be appropriately configured, for example, by providing an appropriately shaped portion of inward-facing surface of the deformable finger so that it corresponds to, or matches with, a correspondingly shaped outer peripheral surface of an injection pen body, or more particularly and preferably, an outer peripheral surface portion of the dose-setting wheel surface. For example, the portion of inward-facing surface of the elastically radially deformable fingers can be moulded into an appropriate shape, such as a concave or arcuate shape, or alternatively, include one or more layers of additional material located on the portion of inward-facing surface of the deformable finger, such as an elastomer in an appropriate thickness, in which the elastomer is compressible so as to adapt to the shape of an outer peripheral surface of the injection pen body, and/or dose-setting wheel.

According to another aspect, each finger of the plurality of elastically radially deformable fingers has a portion of inward-facing surface which is sloped from a proximal point to a distal point, along at least a portion of a length of the elastically radially deformable finger, to conform to an outer peripheral surface of an injection pen body and/or dose-setting wheel, when the attachment system is mounted onto an injection pen, and the second annular body is in the second position.

According to another aspect, the second annular body has at least a first portion of an inward-facing surface which is configured to engage with an outward facing surface of the first annular body. The use of the term “engagement” in this context should be understood to mean that the inward-facing surface of the at least first portion of the second annular body physically engages in surface-to-surface contact with the outward facing surface of the first annular body, either directly or indirectly, but preferably directly. The at least first portion of inward-facing surface of the second annular body can be configured in a variety of ways to perform this function, for example, by shaping of one or more portions of the second annular body, such as by moulding, and/or by providing, on the second annular body, additional layers of material, whereby the moulding and/or provision of additional layers can be shaped to provide one or rims, ridges, grooves, projections, and the like.

According to another aspect, the first portion of inward-facing surface configured to engage with an outward facing surface of the first annular body, is an annular shoulder.

According to another aspect, in the first position, the annular shoulder is configured, and/or shaped, to provide a distal facing surface which engages with a proximal facing surface of a nose portion located at the distal, or second, end of the first annular body, and the distal facing surface of the annular shoulder is free to rotate about the central longitudinal axis and against the proximal facing surface of the nose portion.

According to another aspect, the annular shoulder of the second annular body is configured, and/or shaped, to provide an inward facing portion which engages with an outward facing and inward sloping surface of the first annular body. Such an inward facing portion of the annular shoulder, could be, for example, a ridge, or a dihedral forming an apex facing radially inwardly towards the central longitudinal axis, or a rounded or convex proximally facing surface provided on the annular shoulder. Conversely, the outward facing, and inward sloping, surface of the first annular body will generally extend from a proximal area of each elastically deformable finger, in a distal direction, and be defined by a first outer diameter of the elastically deformable finger, and terminate at a distal location of the finger having a second outer diameter, which is smaller than the first outer diameter of the elastically deformable finger.

According to another aspect, when moving from the first position to the second position, the annular shoulder of the second annular body exerts an inward facing radial force onto the outward facing and inward sloped surface of the first annular body, thereby causing an inward facing surface of the first annular body to be moved inwardly toward the central longitudinal axis. In this way, relative movement of the second annular body with respect to the first annular body, from the first position into the second position, for example, from a distal position to a proximal position, causes a reduction in the diameter of the inner central bore, as the annular shoulder of the second annular body engages with the outward facing and inward sloped surface of the first annular body, for example moving from a relative distal position of the second annular body to a relative proximal position of the second annular body, with respect to the first annular body.

According to another aspect, the second annular body has at least a second portion of an inward-facing surface which is configured, and/or shaped, to engage with an outward facing surface of the first annular body. In a manner similar to the first portion of inward-facing surface of the second annular body, the second inward-facing surface of the second annular body is generally spaced apart from the first portion of inward-facing surface of the second annular body, and is configured to form as an abutting stop for movement of the second annular body against the first annular body, in a proximal direction. The lengthwise separation along a longitudinal axis of the attachment system, between the first inward-facing surface and the second inward-facing surface of the second annular body, is therefore configured to limit the extent to which the second annular body may be moved in a proximal direction, and thereby define the extent of movement of the second annular body from the first position into the second position.

According therefore to another aspect, the second portion of inward-facing surface configured to engage with an outward facing surface of the first annular body is an annular ridge located proximally of the annular shoulder, and adjacent to a proximal end, of the second annular body.

According to another aspect, when moving from the first position to the second position, the annular ridge engages with a helical trough provided on the outward facing surface of the first annular body.

According to another aspect, the helical trough comprises at least one, or a plurality of spires or full revolutions about and along, the central longitudinal axis.

According to another aspect, in the second position, a proximal end of the second annular body abuts a distal end of the first annular body.

According to another aspect, in the second position, the annular ridge of the second annular body comprises a proximal facing and inward sloping surface in stopping abutment with a corresponding distal facing and inward sloping surface of the first annular body.

According to another aspect, the length of the second annular body is less than the length of the first annular body.

According to another aspect, the length of the second annular body is substantially equal to the length of an elastically deformable finger of the first annular body.

According to another aspect, the first position is the unlocked position of the attachment system.

According to another aspect, the second position is the locked position of the attachment system.

According to another aspect, there is also provided an injection monitoring module adapted and configured to be mounted on an injection pen comprising:
an attachment system as substantially described herein; and
a monitoring housing comprising an electronic circuitry component, the monitoring housing being configured to be axially movable within the bore of the first annular body of the attachment system along the central longitudinal axis during operation of the injection monitoring module.

Accordingly, the monitoring housing may comprise a distal body and a proximal cover, the housing being configured and adapted to be mounted co-axially at least partially within the inner central bore of the attachment system. The monitoring housing, as indicated above, may comprise an electronic circuitry component, such as a microcontroller located on an electronic circuit board, the electronic circuit board being held within the housing. The monitoring housing may also advantageously comprise further components, such as:
an autonomous power supply connected to the electronic circuit board;
an activation switch configured to be activated by movement of the proximal housing cover from a first, non-contact position to a second, contact position enabling power to be supplied to the electronic circuit board, or to wake-up one or more sensors connected to the electronic component board;
one or more sensors, such as one or more magnetometers, accelerometers, gyroscopes, and the like, connected to the microcontroller, wherein the microcontroller is configured to process signals received from the one or more sensors to identify parameters and conditions associated with operation of the injection pen system.

As indicated above, the housing of the injection monitoring module may comprise a distal body and a proximal cover The distal body is typically and generally has an open proximal end and a closed distal end, in the manner of a cup or a bowl, and the proximal cover may present a substantially flat or planar surface, or alternatively a domed or convex shape, or alternatively again, an incurved or concave shape. The distal body is shaped and configured to receive, and/or hold, the electronic circuit board within the distal body, or within an inner volume formed by the distal body and proximal cover when distal body and cover are assembled together. The proximal cover and distal body are typically assembled together to form this innner volume, for example, by gluing, clipping, snap-fitting or welding the distal body and proximal cover together, typically at one or more points around an each respective circumference of the open distal body and the proximal cover. The proximal cover, and/or distal body, may comprise one or more portions of transparent or translucent material, for example formed as a window, light guide or light tunnel, and/or may sandwich a ring of such transparent or translucent material. Said window, light guide or light tunnel is usually configured to allow, and/or guide, the passage of visible light emanating from a visible light emission source connected to, and/or located in or on the electronic circuit board, such as a LED, to outside of the housing, such that said emitted light becomes visible to the user. The emitted light is commonly used to indicate a particular status and/or a condition of the injection monitoring module, for example, correct mounting and or positioning of the injection monitoring module relative to the injection pen, ready to set a dose, ready to inject, injection completed, ready to dismount, and the like.

As mentioned above, the microcontroller is located on an electronic circuit board, the electronic circuit board being held within the housing formed by the distal body and proximal cover. The microcontroller typically comprises one or more subcomponents, such as a processor, an internal clock, one or more data registers, and/or a non-volatile memory, and is usually programmable or capable of processing a set of instructions, excuting programmed code, and/or effecting calculations, in order to process information, and/or data and/or electrical signals from the other components connected to, or integrated into, the electronic component board. Such microcontrollers are generally well known per se.

The injection monitoring module may comprise a data storage connected to the microcontroller, and the microcontroller may be configured to calculate, and/or store data relating to an operation condition or status of the injection onto which it is mounted via the attachment system. For the purposes of the present, the data storage can be one of many possibilities, including data registers integrated into the microcontroller described above, and/or volatile and/or non-volatile memory, either integrated into the microcontroller or formed as a separate functional unit and connected to the microcontroller via the electronic component board.

As indicated above, the injection monitoring module may also comprise an autonomous power supply connected to the electronic circuit board. Typically, this can be, for example, a simple battery, such as a button or coin-shaped battery, for example a lithium ion battery, as known in the art. Alternatively, the battery can be rechargeable, again as known per se in the art.

The attachment system, and an injection monitoring module adapted and configured to be mounted on an injection pen comprising such an attachment system, will now be described in greater detail with regard to the figures in which:

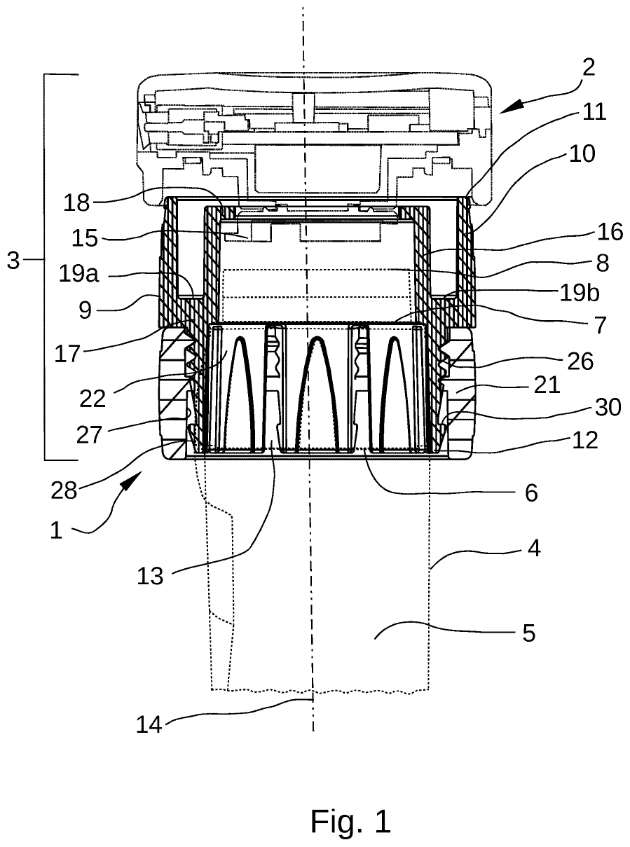

FIG. 1 is a schematic cross-section of the attachment system, in association with an injection monitoring housing to form an injection monitoring module, when mounted on an injection pen;

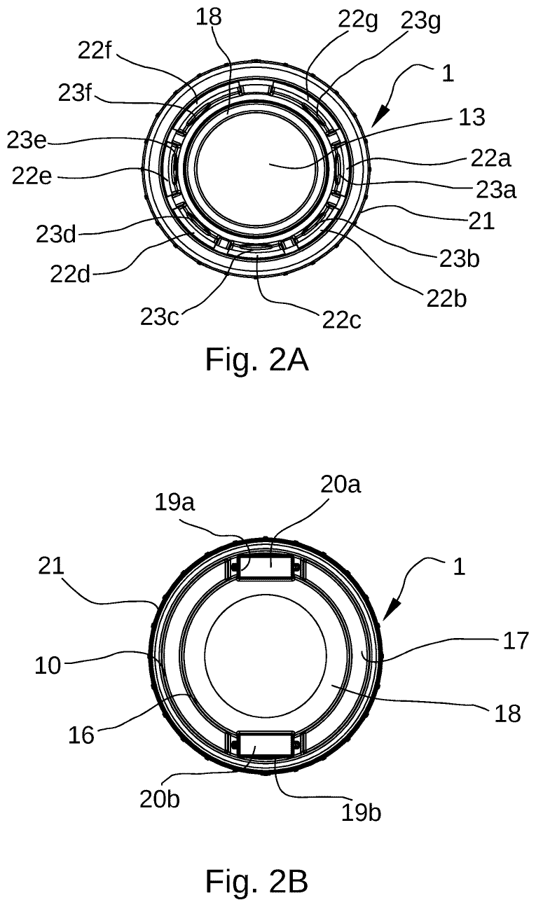

FIG. 2A is a schematic axial view representation of the attachment system of FIGS. 1 and 2 through a central longitudinal bore, from a distal end of the attachment system;

FIG. 2B is a schematic axial view representation of the attachment system of FIGS. 1 and 2 through a central longitudinal bore, from a proximal end of the attachment system.

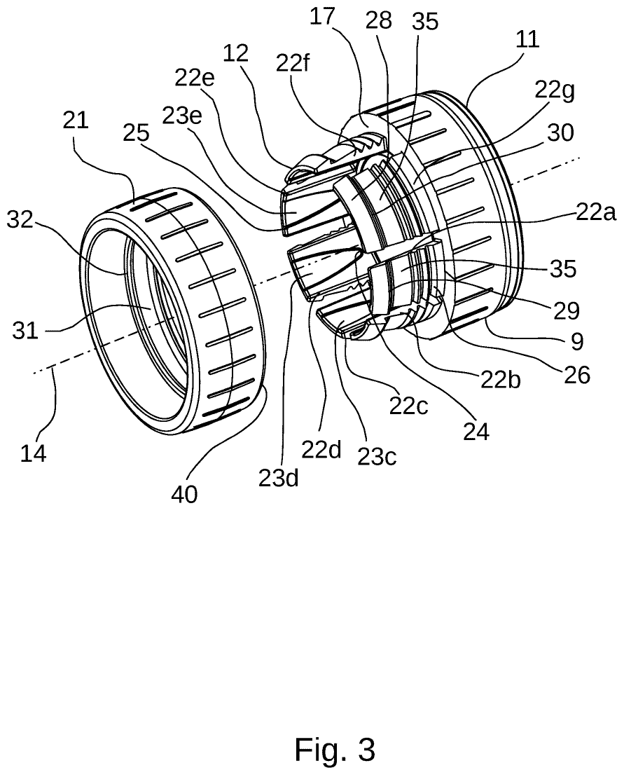

FIG. 3 is a schematic exploded perspective representation of the attachment system of FIG. 1;

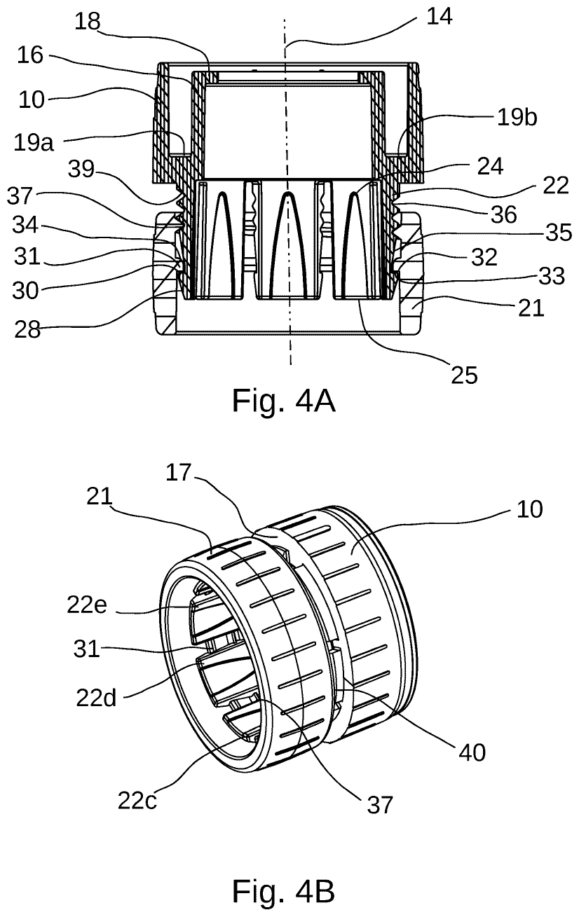

FIG. 4A illustrates a schematic cross-sectional representation of the attachment system of FIGS. 1 and 2 in an assembled, first position;

FIG. 4B illustrates a schematic perspective representation of the attachment system of FIGS. 1 and 2 in the assembled, first position;

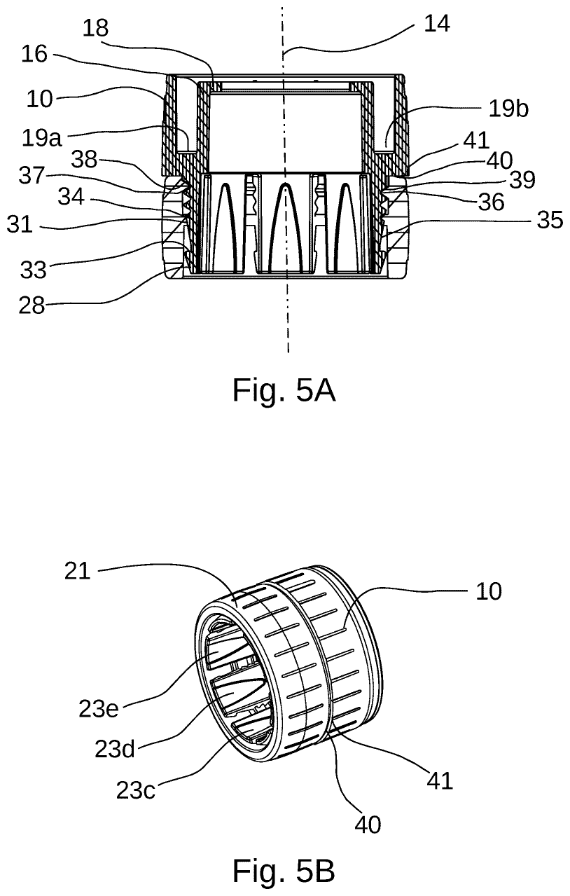

FIG. 5A illustrates a schematic cross-sectional representation of the attachment system of FIGS. 1 and 2 in an assembled, second position;

FIG. 5B illustrates a schematic perspective representation of the attachment system of FIGS. 1 and 2 in the assembled, second position.

### DETAILED DESCRIPTION

Turning now to FIGS. 1, 2A and 2B, an attachment system (1) is shown, associated with an injection monitoring housing (2). Together, the attachment system and injection monitoring housing form an injection monitoring module (3), and are mounted, as illustrated in FIG. 1, onto an injection pen (4), comprising a pen body (5), a dose setting wheel (6) located at a proximal end (7) of the pen body (5), and injection actuation button (8) for effecting an injection. Such injection pens are known per se and may vary in body diameter, length and functioning, depending on the injection pen manufacturer.

The attachment system (1) comprises a first annular body (9) having a length and a diameter, and comprising a first annular wall (10) extending along the length from a first end (11) to a second end (12). The overall length of the first annular body (9) is generally greater than the diameter of the first annular body (9). The first annular wall (10) of the first annular body (9) also defines an inner central bore (13) extending along a length of the attachment system (1), the central bore (13) having a central longitudinal axis (14). Generally, the length of the annular wall (10) of the first annular body (9) will be dimensioned such that the central bore (13) extends in a proximal direction beyond the proximal end (7) of the injection pen when the attachment system is mounted on the pen, and defines a bore volume adapted to receive the actuation button (8) of the injection pen (4), and yet still provide sufficient volume to allow for translational, and/or rotational, movement of the injection monitoring housing (2) within the bore (13), around and/or along, the central longitudinal axis (14). As will be understood from the foregoing, the central bore (13) therefore also receives at least part, for example, a distal part (15), of the injection monitoring housing (2), which is mounted co-axially with the attachment system, and is configured, with regard to the attachment system, to enable, for example, a translational and/or rotational movement of the injection monitoring housing (2) within the bore (13) of the first annular body (9) of the attachment system (1), as is known per se from the previously described and known injection monitoring modules. In the example shown in the figures, the first annular body (9) also comprises a second annular wall (16), situated radially inwardly of the first annular wall (10), and connected to the first annular wall (10) by a shoulder (17) extending radially inwardly from the first annular wall (10). The second annular wall (16), radially spaced apart from the first annular wall (10), and extends from the shoulder (17) in a proximal direction from a first, or distal end, to a second, or proximal end. The overall length of the second annular wall (16) is less than the length of the first annular wall (10) from the shoulder (17) to the first end (11) of the first annular wall (10). As illustrated in FIGS. 1, 2A and 2B, the second annular wall (16) further comprises a radially inwardly projecting shoulder (18) defining an opening having a diameter which is smaller than the diameter of the central bore (13). This opening serves to receive, but also to limit translational axial movement of, a distal part of the injection monitoring housing (2) along the central longitudinal axis (14) of the bore (13). The first annular wall (10) and the second annular wall (16) define an inner radial volume, at least a part of which can appropriately be configured or shaped to form at least one or more housings (19a, 19b) to receive one or more dipole magnets (20a, 20b), as illustrated for example in FIG. 2B, or a ring-shaped dipole ou multipolar magnet. Such a magnet holder housing (19) or housings (19a, 19b) are useful to receive the magnet or magnets (20a, 20b) and provide a magnetic field for the injection monitoring module (2) when the electronic circuitry of the injection monitoring housing comprises at least one magnetometer. If such magnet holder housings (19a, 19b) are each provided with a corresponding dipole magnet (20a, 20b), each magnet can be located in its respective magnet holder housing (19a, 19b) in a respective head-to-tail configuration, i.e. a first dipole magnet (20a) would be located in a first housing (19a) with the N-pole facing in a proximal direction and the S-pole facing in distal direction, and a second dipole magnet (20b) would be located in a second housing (20b) with the N-pole facing in distal direction and the S-pole facing in a proximal direction.

The attachment system also comprises a second annular body (21), mounted coaxially on, and around, the second end (12) of the first annular body (9). The second annular body (21) is configured to be movable relative to the first annular body (9), from a first position to a second position. In the first position, the second annular body (21) exerts no inward radial force on at least one portion of the annular wall (10) of the first annular body, and in the second position, the second annular body (21) exerts an inward radial force on said at least one portion of the first annular wall (10). When moving the second annular body (21) from the first position to the second position, an inward radial force is applied to the at least one portion of the first annular wall (10) causing a reduction in the diameter of the at least one portion of the first annular wall (10). FIGS. 3A and 3B illustrate the relative positions of the second annular body (21) and the first annular body (9) of the attachment system (1), in the first, or unlocked, position, whereas FIGS. 1, 5A, and 5B illustrate the relative positions of the second annular body (21) with respect to the first annular body (9) in the second, or locked, position. To all extents and purposes, as illustrated herein, the first position is an unlocked position of the attachment system (1). In the first, or unlocked position, the second annular body (21) is free to rotate about the first annular body (9), without exerting any inwardly directed radial force or pressure onto the first annular body (9). As a result, the diameter of the central bore (13) is unchanged. To all extents and purposes, as illustrated herein, the second position is a locked position of the attachment system (1). In the second, or locked position, the second annular body (21) is locked against rotation in a direction of rotation, for example, a counter-clockwise direction, which generates application of an inward radial force onto a portion of first annular (10) wall of the first annular body (9). The second annular body (21) is also locked against movement relative to the first annular body (9) in a proximal direction. It follows, as a result of the application or radial inwardly-directed force, that in the second, or locked position, the diameter of the central bore (13) is reduced compared to the diameter of the central bore (13) when the second annular body (21) is in the first, unlocked position.

FIGS. 3, 4A, and 5A illustrate in greater detail a means for achieving the reduction in diameter on application of a radially inwardly directed force to the first annular wall (10) of the first annular body (9) by the second annular body (21), when moving from the first position to the second. Looking at FIG. 3 in more detail, an unassembled presentation of the components of the attachment system are illustrated in an exploded perspective view.

This view shows the first annular body (9) and the second annular body (21), in the unassembled state, i.e. before the second annular body is mounted to, or assembled with, the first annular body (9). The first annular wall (10) of the first annular body (9) is visible, and as are the first, or proximal (11), and second, or distal (12), ends of the first annular wall (10).

The first annular body comprises a number of elastically and radially deformable fingers (22a, 22b, 22c, 22d, 22e, 22f, 22g) which extend towards the second end (12) from the shoulder (17). In the example shown there are 6 elastically and radially deformable fingers, but the number of fingers (22a, 22b, 22c, 22d, 22e, 22f, 22g) can be adjusted when shaping or forming the first annular body (9), e.g. via injection moulding or additive manufacturing, depending on the desired degree of deformation required, and/or the desired degree in reduction of diameter of the inner bore (13). The plurality of elastically radially deformable fingers (22a, 22b, 22c, 22d, 22e, 22f, 22g) is distributed radially about the central longitudinal axis (14), preferably in an equally spaced manner, one from its adjacent or neighbouring, deformable finger (22). Each finger (22) of the plurality of elastically radially deformable fingers (22a, 22b, 22c, 22d, 22e, 22f, 22g) has a portion of inward-facing surface (23a-23g) configured to provide surface-engaging contact with an outer peripheral surface of an injection pen body (5), and in particular, with the outer peripheral surface of the dose setting wheel (6), when the attachment system (1) is mounted onto the injection pen (4), and the second annular body (21) is in the second, or locked, position. As illustrated in FIG. 3, for example, each finger of the plurality of elastically radially deformable fingers (22a-22g) has a portion of inward-facing surface (23a-23g) which is sloped from an each respective proximal point (24) to an each respective distal point (25), along at least a portion of a length of the elastically radially deformable finger (22a-22g), to conform to the outer peripheral surface of the dose setting wheel (6) of the injection pen (4) when the attachment system (1) is mounted onto the injection pen (4), and the second annular body (21) is in the second, or locked, position. The use of the word “sloped”, in this context, refers to a shaping or dimensioning of the elastic fingers to provide the inward-facing engagement, or contact, surfaces (23a-23g).

The shaping or dimensioning of the elastically deformable fingers (23a-23g) can be achieved, for example, by reducing the thickness of each finger from the proximal point (24) towards the distal point (25) of the finger (22). Alternatively, and/or additionally, the shaping or dimensioning of the elastically deformable fingers (23a-23g) can be achieved by adding a layer of friction increasing material, such as an elastomer, from the proximal point (24) to a distal point (25) of the finger (22). Irrespective of the particular mode of execution, the inward-facing engagement, or contact, surfaces (23a-23g) can advantageously be shaped to correspond to similarly shaped outer peripheral surfaces present on the dose setting wheel (6) of the injection pen (4). Each deformable finger (22a-22g) of the first annular body (9) has an outward facing surface (26) which can be engaged by an inward facing surface (27) of the second annular body (21). For example, and as illustrated in FIG. 3, each deformable finger (22a-22g) comprises a distal end, radially outwardly, sloping portion (28), in which the slope extends radially outwards from the distal end (12) of the first annular wall (10), and corresponding finger (22a-22g), in the direction of the proximal end (11), to form an inverted truncated cone area, or nose portion. The proximal end (29) of the inverted, radially outwardly, sloping nose portion (28) forming the truncated cone area provides a shoulder (30), which projects radially outwardly from the first annular wall (10), and on which the second annular body (21) rests when the second annular body (21) is assembled with, or mounted to, the first annular body (9), for example, by pushing the second annular body (21) onto and over the inverted, radially outwardly, sloping nose portion (28) forming the truncated cone area. FIGS. 4A and 4B illustrate in greater detail the relative positions of the assembled first annular body (9) and second annular body (21) in the first, or unlocked, position.

The second annular body (21) has at least a first portion of an inward-facing surface, which is configured to engage with an outward facing surface (26) of the first annular body (9). The first portion of the inward-facing surface of the second annular body (21) is an annular shoulder (31), which is configured to provide a distal facing surface (32), said distal facing surface (32) engaging, when the second annular body (21) is mounted to the first annular body (9), with a proximal facing surface (33) of the shoulder (30) of the nose portion (28). In the first, or unlocked, position, the distal facing surface (32) of the annular shoulder (31) is free to rotate about the central longitudinal axis (14), and against the proximal facing surface (33) of the shoulder (30) of the nose portion (28), without exerting any radially inwardly directed force or effort. In the first, or unlocked, position therefore, the second annular body does not cause elastic deformation of the fingers (22a-22g).

The annular shoulder (31) of the second annular body (21) is also provided with a suitably shaped or configured inwardly facing surface (34), such as a convex surface, which engages with an outward facing sloping portion (35) provided on the outer surface of each elastically deformable finger (22a-22g) of the first annular wall (10) of the first annular body (9). The outward facing sloping portion (35) of the elastically deformable fingers (22a-22g) extends proximally from the proximal facing surface (33) of the shoulder (30) of the nose portion (28), and has an outer diameter, at that distal location, which is smaller than a corresponding outer diameter located proximally and spaced apart from said proximal facing surface (33). The sloping portion (35) of the elastically deformable fingers (22a-22g) thus presents an ever increasing surface contact obstacle to the inwardly facing surface (34) of the annular shoulder (31), as the second annular body (21) is moved from the first, unlocked, position into the second, locked, position. Furthermore, when moving the second annular body (21) from the first, unlocked, position to the second, locked position, the annular shoulder (31) of the second annular body (21) exerts an inward facing radial force onto the outward facing sloping surface portion (35) of the first annular wall (10), thereby causing an inward facing surface of the first annular wall (10) to be moved inwardly toward the central longitudinal axis (14), causing a reduction in diameter of the central bore (13), due to the elasticity of the elastically deformable fingers (22a-22g).

Additionally, the outer surface of the elastically deformable fingers (22a-22g) of the first annular wall (10) is provided with a helical grooved track (36) which is located proximally of the sloping portion (35). The helical grooved track or trough (36) receives, cooperates with, guides and assists in stabilising movement of the second annular body (21) during movement from the first, unlocked position to the second, locked position, and vice-versa, and in particular, receives a second portion of an inward-facing surface of the second annular body formed, or shaped, as an annular ridge (37), which is located proximally of the annular shoulder (31), and adjacent to a proximal end (38) of the second annular body (21). The helical grooved track or trough (36), comprises at least one, or a plurality of spires or full revolutions about and along, the central longitudinal axis (13). The annular ridge (37) of the second annular body (21) engages with, and is received by, the helical grooved track or trough (36)

In order to move the second annular body (21) from the first, or unlocked, position as illustrated in FIGS. 4A and 4B, to the second, or locked, position, as illustrated in FIGS. 5A and 5B, the second annular body (21) is rotated in an anti-clockwise direction about the central longitudinal axis (14) and about the first annular body (9). The annular ridge (37) engages with the helical grooved track (37), causing the annular ridge (36), and correspondingly, the whole second annular body (21), to move in a proximal direction towards the shoulder (17) of the first annular body (9). As the second annular body (21) is moved in the proximal direction, the inwardly facing surface (34) of the annular shoulder (31) bears against the sloping portion (35) of the first annular body (9), thereby pushing the elastically deformable fingers (22a-22g) radially inwardly, and reducing the diameter of the bore (13). As will be understood, when the attachment system (1) is pushed onto and around the dose setting wheel (6) of an injection pen system (4), and the second annular body (21) moved from the first, unlocked position, to the second, locked position, the reduction in diameter of the bore between the radially inwardly moved elastically deformably fingers (22a-22g) causes those fingers to engage with the outer peripheral surface of the dose setting wheel (6), thereby fixing the attachment system in position on, and around the dose setting wheel (6). The second, or locked, position is achieved when the annular ridge (37) has reached the limit of allowable proximal travel prescribed by the helical grooved track (36), and in particular when a proximal facing and inward sloping surface (38) of the annular ridge (37) of the second annular body (21) moves into stopping abutment with a corresponding distal facing and inward sloping surface (39) of the first annular body (9). As can be seen from FIGS. 5A and 5B, in the second, or locked, position, a proximal end (40) of the second annular body (20) abuts a distal end (41) of the shoulder (17) of the first annular body (9).

In order to dismount the attachment system (1) from an injection pen (4), the reverse sequence of operations is carried out. The second annular body (21) is rotated in a clockwise direction about the central longitudinal axis (14) and the first annular body (9). This causes the proximal annular ridge (37) of the second annular body to engage with the helical grooved tracks (36) of the first annular body (9), thereby moving the second annular body (21) in a distal direction. As the second annular body (21) moves in the proximal direction, the radially inwardly constraining engagement of the annular shoulder (31) lessens against the sloping portion (35) of the elastically deformable fingers (22a-22g), which in turn reduces the surface contact of the inward-facing surface portions (23a-23g) with the dose setting wheel, and allows the elastically deformable fingers (22a-22g) to move back to their initial position. The diameter of the central bore (13) of the first annular body (9) thus returns to its initial configuration. Once the second annular body (21) has returned to the first, or unlocked, position, in which the distal facing surface (32) of the annular shoulder (31) is once again in surface contact with the shoulder (30) of the sloping nose portion (28) of the first annular body (9), said annular shoulder (31) of the second annular body (21) is free to rotate without applied constraint to the fingers (22a-22g), around the first annular body (9) and the central longitudinal axis (14). As no inwardly radial constraint is being applied to the fingers (22a-22g) in the first, or unlocked, position, the attachment system can be separated from the injection pen (4). In the manner described an attachment system for an injection monitoring device is provided which is both easy to mount, and easy to remove, requiring little physical effort to correctly position and safely fix the attachment system to the dose setting wheel of any injection pen having such a dose setting wheel, irrespective of its relative dimensions, and also ensure by the same mechanisms that any associated injection monitoring housing is also correctly positioned.

1. Attachment system configured and adapted for mounting an injection monitoring module on an injection pen comprising:
a first annular body having a length and a diameter, and comprising an annular wall extending along the length from a first end to a second end, the length being greater than the diameter, the annular wall defining an inner central bore extending along the length and having a central longitudinal axis;
a second annular body mounted coaxially on, and around, the second end of the first annular body;
the second annular body being configured to be movable relative to the first annular body, from a first position to a second position;
wherein,
in the first position, the second annular body exerts no inward radial force on at least one portion of the annular wall of the first annular body, and
in the second position, the second annular body exerts an inward radial force on the at least one portion of the first annular wall,
wherein the inward radial force applied to the at least one portion of the first annular wall operates a reduction in the diameter of the at least one portion of the first annular wall.

2. Attachment system according to claim 1, wherein the annular wall of the first annular body comprises a plurality of elastically radially deformable fingers extending towards the second end.

3. Attachment system according to claim 2, wherein the plurality of elastically radially deformable fingers are distributed radially about the central longitudinal axis.

4. Attachment system according to claim 2 wherein the elastically radially deformable fingers are deformable radially inwardly toward the central longitudinal axis, when the second annular body exerts an inward radial force on the at least one portion of the first annular wall.

5. Attachment system according to claim 2,
wherein each finger of the plurality of elastically radially deformable fingers has a portion of inward-facing surface configured to provide surface-engaging contact with an outer peripheral surface of an injection pen body when the attachment system is mounted onto an injection pen, and the second annular body is in the second position.

6. Attachment system according to any claim 2, wherein each finger of the plurality of elastically radially deformable fingers has a portion of inward-facing surface which is sloped from a proximal point to a distal point, along at least a portion of a length of the elastically radially deformable finger, to conform to an outer peripheral surface of an injection pen body when the attachment system is mounted onto an injection pen, and the second annular body is in the second position.

7. Attachment system according to claim 1, wherein the second annular body has at least a first portion of an inward-facing surface which is configured to engage with an outward facing surface of the first annular body.

8. Attachment system according to claim 7, wherein the first portion of inward-facing surface configured to engage with an outward facing surface of the first annular body, is an annular shoulder.

9. Attachment system according to claim 8, wherein, in the first position, the annular shoulder is configured to provide a distal facing surface which engages with a proximal facing surface of a nose portion located at a distal end of the first annular body, and the distal facing surface of the annular shoulder is free to rotate about the central longitudinal axis and against the proximal facing surface of the nose portion.

10. Attachment system according to claim 8, wherein the annular shoulder of the second annular body is configured to provide an inward facing portion which engages with an outward facing and inward sloping surface of the first annular body.

11. Attachment system according to claim 8, wherein, when moving from the first position to the second position, the annular shoulder of the second annular body exerts an inward facing radial force onto the outward facing and inward sloped surface of the first annular body, thereby causing an inward facing surface of the first annular body to be moved inwardly toward the central longitudinal axis.

12. Attachment system according to claim 1, wherein the second annular body has at least a second portion of an inward-facing surface which is configured to engage with an outward facing surface of the first annular body.

13. Attachment system according to claim 12, wherein the second portion of inward-facing surface configured to engage with an outward facing surface of the first annular body is an annular ridge located proximally of the annular shoulder, and adjacent to a proximal end, of the second annular body.

14. Attachment system according to claim 13, wherein, when moving from the first position to the second position, the annular ridge engages with a helical trough provided on the outward facing surface of the first annular body.

15. Attachment system according to claim 13, wherein the helical trough comprises at least one, or a plurality of spires or full revolutions about and along, the central longitudinal axis.

16. Attachment system according to claim 1, wherein, in the second position, a proximal end of the second annular body abuts a distal end of the first annular body.

17. Attachment system according to, claim 12, wherein, in the second position, the annular ridge of the second annular body comprises a proximal facing and inward sloping surface in stopping abutment with a corresponding distal facing and inward sloping surface of the first annular body.

18. Attachment system according to claim 1, wherein the length of the second annular body is less than the length of the first annular body.

19. Attachment system according to claim 1, wherein the length of the second annular body is substantially equal to the length of an elastically deformable finger of the first annular body.

20. Attachment system according to any claim 1, wherein the first position is the unlocked position of the attachment system.

21. Attachment system according to claim 1, wherein the second position is the locked position of the attachment system.

22. Injection monitoring module adapted and configured to be mounted on an injection pen comprising:
an attachment system according to claim 1;
a monitoring housing comprising an electronic circuitry component, the monitoring housing being configured to be axially movable within the bore of the first annular body of the attachment system along the central longitudinal axis during operation of the injection monitoring module.
